(19) United States
(12) Patent Application Publication
(10) Pub. No.: US 2025/0375297 A1
(43) Pub. Date: Dec. 11, 2025

(54) MONOLITHIC BASEPLATE

(71) Applicant: HOWMEDICA OSTEONICS CORP., Mahwah, NJ (US)

(72) Inventors: Benjamin DASSONVILLE, Saint Hilaire (FR); Vincent GABORIT, Saint Martin d'Hères (FR); Olivier ZANARDI, Bernin (FR); Quentin BOUVIER, Bernin (FR); François AOUTIN, Corenc (FR); Alexia AUBURTIN, Nice (FR); Gilles HENRY, Le Pont de Claix (FR)

(73) Assignee: HOWMEDICA OSTEONICS CORP., Mahwah, NJ (US)

(21) Appl. No.: 18/876,884

(22) PCT Filed: Jul. 13, 2023
(86) PCT No.: PCT/US2023/070118

§ 371 Date: Dec. 19, 2024

Related U.S. Application Data
(60) Provisional application No. 63/368,419, filed on Jul. 14, 2022.

(51) Int. Cl. A61F 2/30; A61F 2/40

(52) U.S. Cl. CPC A61F 2/30734; A61F 2/4081; A61F 2002/30736

(57) ABSTRACT

Various embodiments of a novel glenoid implant baseplate are disclosed. Provided is a prosthesis for attachment to a glenoid that includes a baseplate body that is a monolithic metallic structure and incorporates a distal surface and a proximal surface, a post extending from the proximal surface, and features associated with the post that can provide orientation stability for a modular bone augment. Also disclosed is a prosthesis for attachment to a glenoid that includes a baseplate body that is a monolithic metallic structure that incorporates an integrally formed metallic bone augment.

### CROSS-REFERENCE TO RELATED APPLICATIONS

This application claims priority to U.S. Provisional Application No. 63/368,419, filed on Jul. 14, 2022, the entire contents of which are incorporated herein by reference.

### FIELD OF DISCLOSURE

The present disclosure generally relates to glenoid implants for shoulder prosthesis.

### BACKGROUND

Existing reverse shoulder glenoid implant baseplates are not adequately compatible with using modular bone augments that are positioned between the glenoid and the baseplate. Thus, improved glenoid implant baseplate design is desired. Additionally, better incorporation of metallic bone augments in reverse shoulder glenoid implant baseplates is also desired.

### SUMMARY

Provided are various embodiments of a prosthesis for attachment to a glenoid that comprises a baseplate body that is a monolithic metallic structure and have features configured for accommodating modular bone augments. The modular bone augments can be bone grafts or metallic augments. Also provided are embodiments of a prosthesis for attachment to a glenoid that comprises a baseplate body that is a monolithic metallic structure that has integrated a metallic bone augment into its structure.

In some embodiments, the baseplate body comprises a distal surface, a proximal surface, a post extending from the proximal surface, and a plurality of fins radially extending from the post at or near where the post meets the proximal surface.

Also provided are other embodiments of a prosthesis for attachment to a glenoid that comprises a baseplate body that is a monolithic metallic structure where the baseplate body comprises a distal surface, a proximal surface, a post extending from the proximal surface, where the post has a tapered conical shape with a taper angle of 5 to 30 degrees whereby its diameter decreases in the proximal direction, and a plurality of fins provided at the tip of the post and radially extending from the post.

Provided is another embodiment of a prosthesis for attachment to a glenoid that comprises a baseplate body that is a monolithic metallic structure where the baseplate body comprises a distal surface, a proximal surface, a post extending from the proximal surface, where the post comprises a wireframe structure having a cylindrical outline and defining an interior volume that is configured as a porous structure.

Also provided is another embodiment of a prosthesis for attachment to a glenoid that comprises a baseplate body that is a monolithic metallic structure where the baseplate body comprises a distal surface, a proximal surface, a post extending from the proximal surface, where the post comprises a hollow structure that defines an interior volume, where the hollow substantially cylindrical structure comprises a plurality of openings providing access to the interior volume.

Provided is another embodiment of a prosthesis for attachment to a glenoid that comprises a baseplate body that is a monolithic metallic structure where the baseplate body comprises a distal surface, a proximal surface opposite from the distal surface, where the proximal surface comprises a bone augment portion that extends away from the distal surface and defines a bone-engaging surface that is spaced apart from the distal surface, and a post extending from the bone augment, where the post comprises an outer surface and a plurality of porous surface portions.

### BRIEF DESCRIPTION OF THE DRAWINGS

The various embodiments of the inventive implant of the present disclosure will be described in more detail in conjunction with the following drawing figures. The structures in the drawing figures are illustrated schematically and are not necessarily intended to show actual dimensions or relative scale.

FIG. 1A is an illustration of an embodiment of the prosthesis according to the present disclosure.

FIG. 1B is an illustration of another embodiment of the prosthesis according to the present disclosure.

FIG. 1C is a close up detailed view illustration of the radially extending fins on the prosthesis of FIG. 1B.

FIG. 1D is a representative sectional view of the prostheses of FIGS. 1A and 1B.

FIG. 2 is an illustration of an example of a modular bone augment that can be used in conjunction with the various embodiments of the prosthesis of the present disclosure.

FIGS. 3A-3B are illustrations of another embodiment of the prosthesis according to the present disclosure.

FIGS. 4A-4B are illustrations of another embodiment of the prosthesis according to the present disclosure.

FIG. 5A is an illustration of another embodiment of the prosthesis according to the present disclosure.

FIGS. 5B-5C are illustrations of another embodiment of the prosthesis according to the present disclosure.

FIG. 5D is an illustration showing an example of an articular component that can be mated to the prosthesis of the present disclosure.

FIGS. 6A-6B are illustrations of another embodiment of the prosthesis according to the present disclosure.

FIG. 6C is a representative sectional view of the prosthesis of FIGS. 6A-6B.

FIGS. 7A-7D are illustrations representing both (i) an embodiment of the prosthesis of FIGS. 6A-6C assembled with a modular metallic bone augment example; and (ii) an embodiment of a prosthesis that has an integrally formed metallic bone augment.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

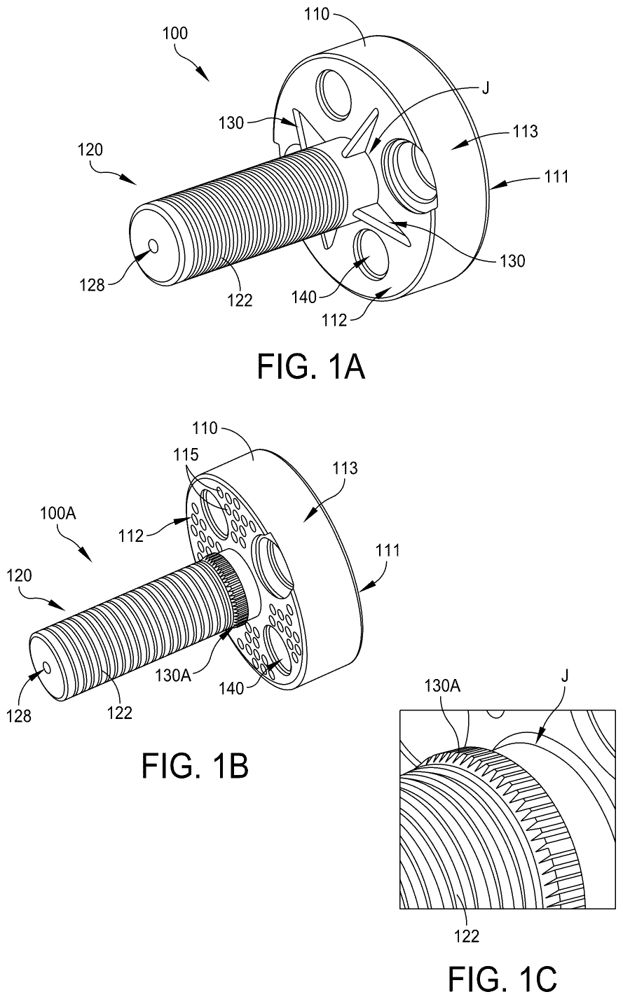

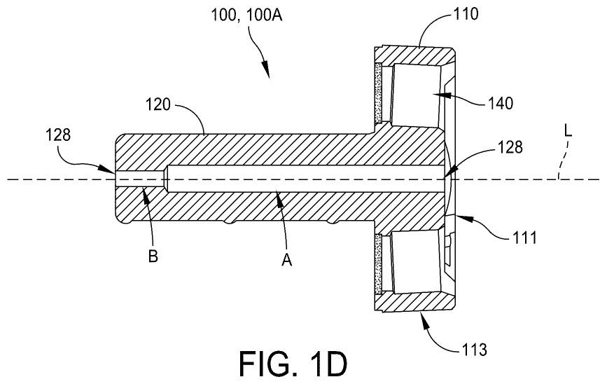

Referring to FIG. 1A, provided is an embodiment of a prosthesis 100 for attachment to a glenoid. The prosthesis 100 comprises a baseplate body 110 that is a monolithic metallic structure. The baseplate body 110 comprises a distal surface 111, a proximal surface 112, and a post 120 extending from the proximal surface 112. Described herein are features of the baseplate body 110 and the post 120 that together provide orientational stability for a modular bone graft used with the prosthesis 100.

In some embodiments, the monolithic metallic structure of the baseplate body 110 can be fabricated by an additive manufacturing process such as 3-D printing. The post 120 is configured to be disposed in a complementary hole or a recess prepared in the glenoid to participate in affixing the prosthesis 100 to the glenoid.

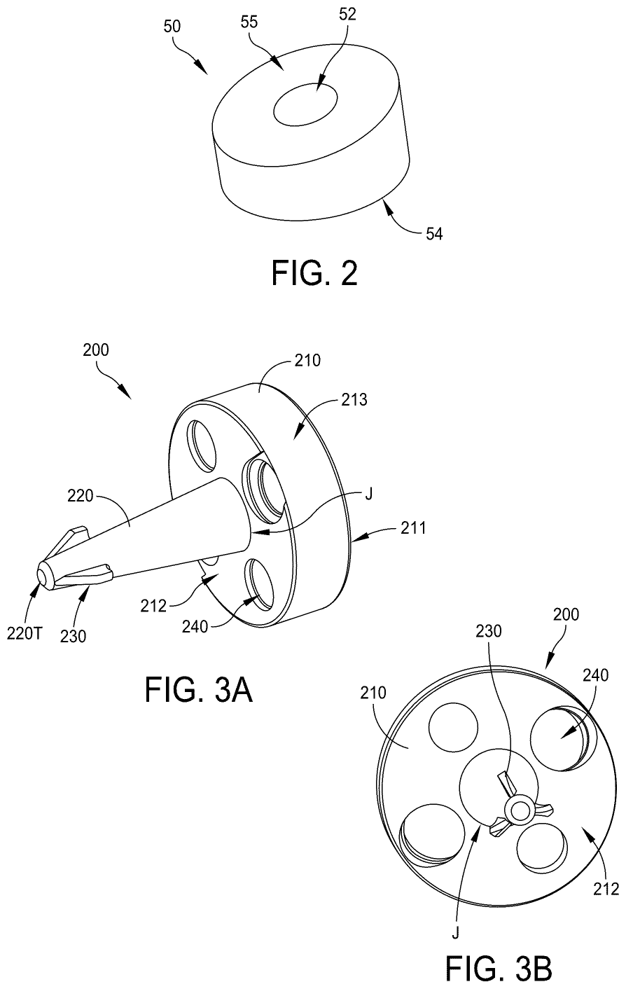

In some embodiments, the proximal surface 112 is configured to engage a modular bone augment, such as the bone augment 50 shown in FIG. 2, and secures the modular bone augment between the baseplate body 110 and the glenoid of a patient. As shown in the example modular bone augment 50, the modular bone augments can have two main surfaces, a first surface 54 and a second surface 55. The first surface 54 engages the proximal surface 112 of the baseplate body and the second surface 55 faces the glenoid surface. The shape and contour of the second surface 55 is configured to fill a void in the deteriorated glenoid. In some applications, the second surface 55 can be patient-matched to more accurately and effectively fill the void in the glenoid. Because of the function of the bone augments 50, the second surface 55 is often angled and/or non-symmetric and require a specific orientation on the baseplate. The bone augments 50 would need to be installed with the prosthesis 100 in a specific orientation to properly augment the bone where voids may exist in the bone.

In some embodiments, as illustrated in the prosthesis 100A shown in FIG. 1B, the proximal surface 112 can be provided with a plurality of dimples 115 to promote bone tissue in-growth at the interface between the proximal surface 112 and a modular bone augment to enhance fixation of the prosthesis 100A after implantation.

The baseplate body 110 can also comprise a plurality of fins 130 that radially extend from the post 120 at or near where the post 120 meets the proximal surface 112. As shown in the illustrated example in FIG. 1A, in some embodiments, the plurality of fins 130 can be located along junction J where the post 120 meets the proximal surface 112 and extend radially along the proximal surface 112. The fins 130 can taper toward the proximal surface 112 as they extend radially outward from the post 120. In some embodiments, the modular bone augment 50 can be configured to receive the fins 130 as the bone augment 50 engage the proximal surface 112. For example, the first surface 54 of the bone augment 50 can be provided with recess or grooves that are positioned to receive the fins 130. The engagement between the fins 130 and the bone augment 50 can secure the bone augment 50 in a desired orientation with respect to the baseplate body 110 so that the required specific orientation of the bone augment 50 can be maintained.

Referring to FIG. 2, the modular bone augment 50 can be configured to be slipped over the post 120 and abut the proximal surface 112 of the baseplate body 110. The bone augment 50 can be provided with a hole 52 that extends through the thickness of the bone augment from the first surface 54 to the second surface 55. The hole 52 is sized to receive the post 120 so that the bone augment 50 can be slipped over the post 120. The bone augment 50 is configured so that the first surface 54 abuts the proximal surface 112 when the bone graft is slipped over the post 120. The second surface 55 is contoured to match the shape and contour of the void in the surface of the glenoid. The modular bone augment 50 can be made of bone graft or porous surgical grade material that is bio-compatible in a human body when implanted. Such porous material can be a metallic material, a ceramic material, or a polymer material. The example of the bone augment 50 shown in FIG. 2 has a generally cylindrical or a disc-like shape but the shape of the bone augment 50 does not have to be so limited. The shape and contour of the bone augment 50 can be varied and can be provided in any shape that is necessary to augment the void in the glenoid as long as the bone augment can be secured to the proximal side of the baseplate body 110 to maintain the desired specific orientation of the bone augment when the prostheses 100, 100A and prevent the bone augment 50 from rotating about the post 120.

In embodiments where the modular bone augment 50 is made of bone graft, when the modular bone augment 50 is installed, its first surface 54 would be in contact with the proximal surface 112 of the baseplate body 110, and the plurality of fins 130 can cut into the bone augment 50 when the bone augment 50 is pressed against the proximal surface 112 and prevent the bone graft 50 from rotating about the post 120.

The plurality of fins 130 on the baseplate can take on a variety of structural configurations as long as they provide a means of preventing unwanted rotation of the modular bone augment 50 after the bone augment 50 is installed. Referring to FIGS. 1B and 1C, in an embodiment of prosthesis 100A, the plurality of fins 130A can be provided on the post 120 and located near the junction J where the post 120 meets the proximal surface 112 but do not touch the proximal surface 112. In the particular example shown in FIGS. 1B and 1C, the fins 130A provided on the post 120 can be configured as short teeth-like forms and can be spaced apart from the junction J.

In some embodiments, the prostheses 100, 100A, can comprise a plurality of openings 140 provided between the post 120 and the baseplate body's periphery for receiving one or more bone screws to help secure the prostheses 100, 100A to the glenoid. Each of the plurality of openings 140 extend through the baseplate body 110 from the distal surface 111 to the proximal surface 112.

In some embodiments, the post 120 can have a generally cylindrical shape whose diameter gradually decreases in the proximal direction.

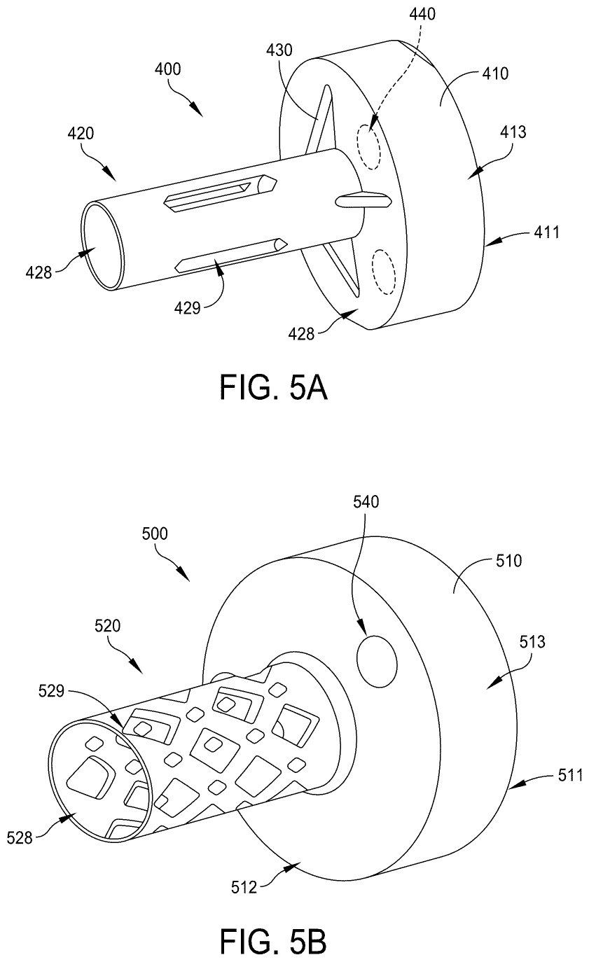

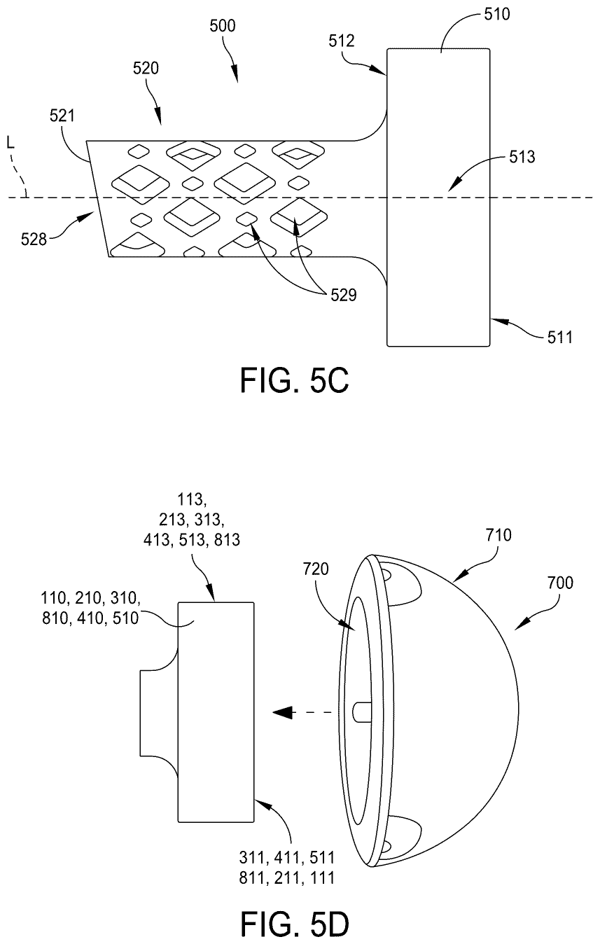

Referring to FIG. 1D, in some embodiments, the post 120 can be configured with a hole or a channel 128 that can assist in aligning an articular component with the prostheses 100, 100A when the articular component is being attached to the baseplate body 110. The channel 128 extends from the distal surface 111 into the post 120 along the longitudinal axis L of the post 120. In some embodiments, as shown in the example in FIG. 1D, the channel 128 can extend through the whole length of the post 120. The channel 128 terminates at the distal surface 111 and has a diameter that is appropriate for snugly receiving a guide pin. In use, a guide pin can be inserted into the channel 128 from the distal surface 311 side so that the guide pin extends out of the channel 128 at the distal surface 111 side. An articular component, such as the example articular component 700 shown in FIG. 5D, that is configured to mate with the baseplate body 110 can be provided with a corresponding hole or a channel that is configured to receive the guide pin that is extending out of the channel 128, so that the guide pin can help align the articular component 700 and the baseplate body 110 as the two components are joined together.

The channel 128 can be a blind hole that extends a predetermined depth into the post 120 so that the guide pin inserted fully into the portion A extends out of the channel 128 an appropriate amount and provide the guiding function for the articular component 700.

In some embodiments, the channel 128 can extend through the whole length of the post 120 so that the channel 128 is open at the distal surface 111 and at the proximal end of the post 120. In such embodiment, the channel 128 can be configured with two portions A and B where the portion A is the portion that opens at the distal surface 111 and receives the guide pin. The portion B can have a smaller diameter than the portion A so that the guide pin is too large to fit into the smaller diameter portion B. This configuration prevents the guide pin from exiting at the proximal end of the post 120 and into the patient's bone. Additionally, having the channel 128 open at both ends allow debris and fluids generated during the manufacturing process of drilling the channel 128 to be evacuated.

Referring to FIGS. 3A-3B, a prosthesis 200 for attachment to a glenoid according to another embodiment is provided. The prosthesis 200 comprises a baseplate body 210 that is a monolithic metallic structure. The baseplate body 210 comprises a distal surface 211, a proximal surface 212, and a post 220 extending from the proximal surface 212. Described herein are features of the baseplate body 210 and the post 220 that provide orientational stability for a modular bone graft used with the prosthesis 200.

In this embodiment, the post 220 comprises a tapered conical shape with a taper angle of about 5 to 30 degrees whereby its diameter decreases in the proximal direction from the baseplate body 210. The tapered shape of the post 220 provides progressive press-fitting engagement with the glenoid where the post 220 can be pressed into the glenoid with some minimum preparation of the glenoid (i.e., reaming and/or drilling) or without any preparation of the glenoid.

The post 220 can also comprise a plurality of fins 230 provided at the tip 220T of the post 220, where the fins 230 radially extend from the post 220. The tip 220T is the end of the post 220 that is furthest away from the baseplate body 210. The fins 230 can be shaped to further enable the ability to press the tapered post 220 into the glenoid with or without any preparation of the glenoid. For example, the leading edges of the fins 230 can be sharp as a blade and raked as shown in FIG. 3A so that the fins 230 can cut into the glenoid and allow the tapered post 220 to be pushed into the glenoid easily.

In some embodiments, the monolithic metallic structure of the baseplate body 210 can be fabricated by an additive manufacturing process such as 3-D printing. The post 220 is configured to be disposed in a complementary hole or a recess prepared in the glenoid to participate in affixing the prosthesis 200 to the glenoid.

In some embodiments, the prosthesis 200 can further comprise a plurality of second set of fins similar to the plurality of fins 130 and 130A in the prosthesis embodiments 100 and 100A, respectively. Such second set of fins radially extend from the post 220 at or near the junction J where the post 220 meets the proximal surface 212. In some embodiments, the plurality of second set of fins are located at the junction J and extend radially along the proximal surface, similar to the fins 130 shown in FIG. 1A. In some embodiments, the plurality of second set of fins are located near the junction J but do not touch the proximal surface 212.

In some embodiments, the prosthesis 200 can further comprise a plurality of openings 240 provided between the post 220 and the baseplate body's periphery for receiving one or more bone screw to secure the prostheses 200 to the glenoid. Each of the plurality of openings 240 extend through the baseplate body 210 from the distal surface 211 to the proximal surface 212.

In some embodiments, the prosthesis 200 can further comprise a modular bone augment 50 configured to be slipped over the post 220 and abut the proximal surface 212 of the baseplate body 210.

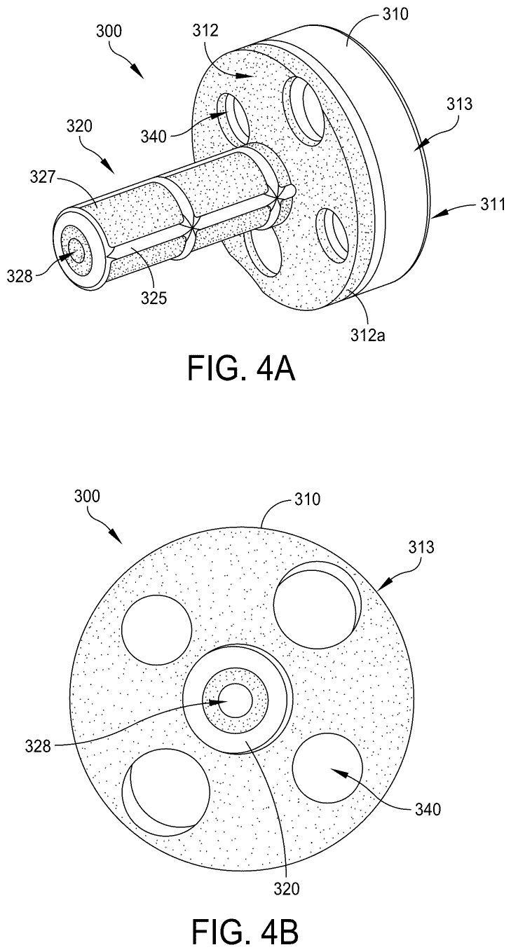

Referring to FIGS. 4A-4B, a prosthesis 300 for attachment to a glenoid according to another embodiment is provided. The prosthesis 300 comprises a baseplate body 310 that is a monolithic metallic structure. The baseplate body 310 comprises a distal surface 311, a proximal surface 312, and a post 320 extending from the proximal surface 312. In this embodiment, the post 320 comprises a wireframe structure 325 having a cylindrical outline and defines an interior volume that is configured as a porous structure 327. Described herein are features of the baseplate body 310 and the post 320 that provide orientational stability for a modular bone augment used with the prosthesis 300.

In some embodiments, the monolithic metallic structure of the baseplate body 310 can be fabricated by an additive manufacturing process such as 3-D printing. The porous structure 327 can act as a scaffold that facilitates bone ingrowth after the prosthesis 300 is implanted into a glenoid.

Both the wireframe structure 325 and the porous structure 327 can be formed from the same metallic material and can be formed concurrently through the additive manufacturing process. This allows the structurally composite (not materially composite as the wireframe structure 325 and the porous structure 327 are made of the same material) configuration for the post 320 in which the two structural portions, the wireframe structure 325 and the porous structure 327, are well integrated. The post 320 is configured to be disposed in a complementary hole or a recess prepared in the glenoid to participate in affixing the prosthesis 300 to the glenoid.

In some embodiments of the prosthesis 300, the porous structure 327 can be configured to occupy the internal volume of the wireframe structure 325 completely to maximize the amount of bone ingrowth into the post 320. In some embodiments, similar to the post 120 in the prostheses 100, 100A, the post 320 can be configured with a hole or a channel 328 that assists in aligning a glenosphere component with the prosthesis 300 during its attachment to the baseplate body 310. The structure and function of the channel 328 is the same as the channel 128 provided in the post 120.

In some embodiments of the prosthesis 300, the proximal surface 312 can be configured with a surface layer 312a that is configured as a porous structure. This provides increased surface area on the prosthesis 300 that can facilitate bone ingrowth after the prosthesis 300 is implanted into a glenoid.

In some embodiments, the cylindrical outline of the wireframe structure 325 is a circular cylinder. In some embodiments, the cylindrical outline of the wireframe structure is a non-circular cylinder. For example, the cylindrical outline is a cylinder having an oval cross-section.

In some embodiments, the prosthesis 300 can further comprise a plurality of fins (similar to the fins 130 illustrated with the prosthesis embodiment 100) that are radially extending from the post 320 at or near where the post meets the proximal surface 312. In some embodiments, the plurality of fins can be located where the post meets the proximal surface 312 and extend radially along the proximal surface. In some embodiments, the plurality of fins are located near where the post meets the proximal surface but do not touch the proximal surface 312.

In some embodiments, the prosthesis 300 can further comprise a plurality of openings 340 provided between the post 320 and the baseplate body's periphery for receiving one or more bone screw to secure the prostheses 300 to the glenoid. Each of the plurality of openings 340 extend through the baseplate body 310 from the distal surface 311 to the proximal surface 312.

In some embodiments, the prosthesis 300 can further comprise a modular bone augment 50 configured to be slipped over the post 320 and abut the proximal surface 312 of the baseplate body 310.

Referring to FIG. 5A, a prosthesis 400 for attachment to a glenoid according to another embodiment is provided. The prosthesis 400 comprises a baseplate body 410 that is a monolithic metallic structure. The baseplate body 410 comprises a distal surface 411, a proximal surface 412, and a post 420 extending from the proximal surface 412. In this embodiment, the post 420 comprises a hollow substantially cylindrical structure that defines an interior volume. The hollow substantially cylindrical structure of the post 420 comprises a plurality of openings 429 providing access to the interior volume. The hollow cylindrical structure of the post 420 further comprises an opening 428 at the proximal end of the post 420. The proximal end of the post 420 being the end furthest away from the baseplate body 410. Described herein are features of the baseplate body 410 and the post 420 that provide orientational stability for a modular bone graft used with the prosthesis 400.

In some embodiments, the monolithic metallic structure of the baseplate body 410 can be fabricated by an additive manufacturing process such as 3-D printing. The post 420 is configured to be disposed in a complementary hole or a recess prepared in the glenoid to participate in affixing the prosthesis 400 to the glenoid.

In some embodiments, the hollow structure of the post 420 can have a substantially cylindrical shape. In some embodiments, the hollow structure of the post 420 can have a circular cylindrical shape. In some embodiments, the hollow structure of the post 420 can have a non-circular cylindrical shape.

In some embodiments, the baseplate body 410 can comprise an opening 428 that extends through the baseplate body 410 and through the full length of the post 420 that provides access to the interior volume of the post 420. The hollow structure of the post 420 can comprise a plurality of openings 429 that also provides access to the interior volume of the post 420. The hollow post 420 can be implanted into glenoid by impacting the hollow post 420 into the glenoid which would require minimum bone preparation. As the prosthesis 400 is impacted into the glenoid, the boney material will fill the interior volume of the hollow post 420. After the prosthesis 400 is fully implanted, the plurality of openings 429 allow the bone tissue inside the post 420 and outside the post 420 to bridge through the openings 429 grow into each other, thus strengthening the fixation of the prosthesis 400 in the bone.

In some embodiments, the interior volume of the post 420 and the openings 429 can be filled with a porous material such as ADAPTIS™ by Wright Medical Group N.V. This filling of the interior volume can be accomplished through the opening 428 at the proximal end of the post 420.

In some embodiments, the prosthesis 400 can further comprise a plurality of fins (similar to the fins 130 shown for the prosthesis embodiment 100) that radially extend from the post 420 at or near where the post meets the proximal surface 412. In some embodiments, the plurality of fins can be located where the post meets the proximal surface 412 and extend radially along the proximal surface 412. In some embodiments, the plurality of fins can be located near where the post meets the proximal surface 412 but do not touch the proximal surface 412.

In some embodiments, the prosthesis 400 can further comprise a plurality of openings 440 provided between the post 420 and the baseplate body's periphery. Each of the plurality of openings 440 extend through the baseplate body 410 from the distal surface 411 to the proximal surface 412 for receiving one or more screw for assisting in securing the prosthesis 400 to the glenoid.

In some embodiments, the prosthesis 400 can further comprise a bone graft 50 configured to be slipped over the post 420 and abut the proximal surface 412 of the baseplate body 410.

Referring to FIGS. 5B and 5C, a prosthesis 500 for attachment to a glenoid according to another embodiment is provided. The prosthesis 500 comprises a baseplate body 510 that is a monolithic metallic structure. The baseplate body 510 comprises a distal surface 511, a proximal surface 512, and a post 520 extending from the proximal surface 512. In this embodiment, the post 520 comprises a hollow substantially cylindrical structure that defines an interior volume.

The hollow substantially cylindrical structure of the post 520 comprises a plurality of openings 529 providing access to the interior volume. The hollow cylindrical structure of the post 520 further comprises an opening 528 at the proximal end of the post 520. The proximal end of the post 520 being the end furthest away from the baseplate body 510. Described herein are features of the baseplate body 510 and the post 520 that provide orientational stability for a modular bone graft used with the prosthesis 500.

In some embodiments, the monolithic metallic structure of the baseplate body 510 can be fabricated by an additive manufacturing process such as 3-D printing. The post 520 is configured to be disposed in a complementary hole or a recess prepared in the glenoid to participate in affixing the prosthesis 500 to the glenoid.

In some embodiments, the hollow structure of the post 520 can have a substantially cylindrical shape. In some embodiments, the hollow structure of the post 520 can have a circular cylindrical shape. In some embodiments, the hollow structure of the post 520 can have a non-circular cylindrical shape.

In some embodiments, the baseplate body 510 can comprise an opening 528 that extends through the baseplate body 510 and through the full length of the post 520 that provides access to the interior volume of the post 520. The hollow structure of the post 520 can comprise a plurality of openings 529 that also provides access to the interior volume of the post 520. The hollow post 520 can be implanted into glenoid by impacting the hollow post 520 into the glenoid which would require minimum bone preparation. As the prosthesis 500 is impacted into the glenoid, the boney material will fill the interior volume of the hollow post 520. After the prosthesis 500 is fully implanted, the plurality of openings 529 allow the bone tissue inside the post 520 and outside the post 520 to bridge through the openings 529 grow into each other, thus strengthening the fixation of the prosthesis 500 in the bone. As shown in FIG. 5C, the edge 521 of the hollow post 520 can be angled at a slant with respect to the longitudinal axis L of the post 520 so that the edge 521 can facilitate the initial protrusion into the glenoid as the hollow post 520 is impacted into the bone.

In some embodiments, the interior volume of the post 520 and the openings 529 can be filled with a porous material such as Adaptis™ by Wright Medical Group N.V. This filling of the interior volume can be accomplished through the opening 528 at the proximal end of the post 520.

In some embodiments, the prosthesis 500 can further comprise a plurality of fins (similar to the fins 130 shown for the prosthesis embodiment 100) that radially extend from the post 520 at or near where the post meets the proximal surface 512. In some embodiments, the plurality of fins can be located where the post meets the proximal surface 512 and extend radially along the proximal surface 512. In some embodiments, the plurality of fins can be located near where the post meets the proximal surface 512 but do not touch the proximal surface 512.

In some embodiments, the prosthesis 500 can further comprise a plurality of openings 540 provided between the post 520 and the baseplate body's periphery. Each of the plurality of openings 540 extend through the baseplate body 510 from the distal surface 511 to the proximal surface 512 for receiving one or more screw for assisting in securing the prosthesis 500 to the glenoid.

In some embodiments, the prosthesis 500 can further comprise a bone graft 50 configured to be slipped over the post 520 and abut the proximal surface 512 of the baseplate body 510.

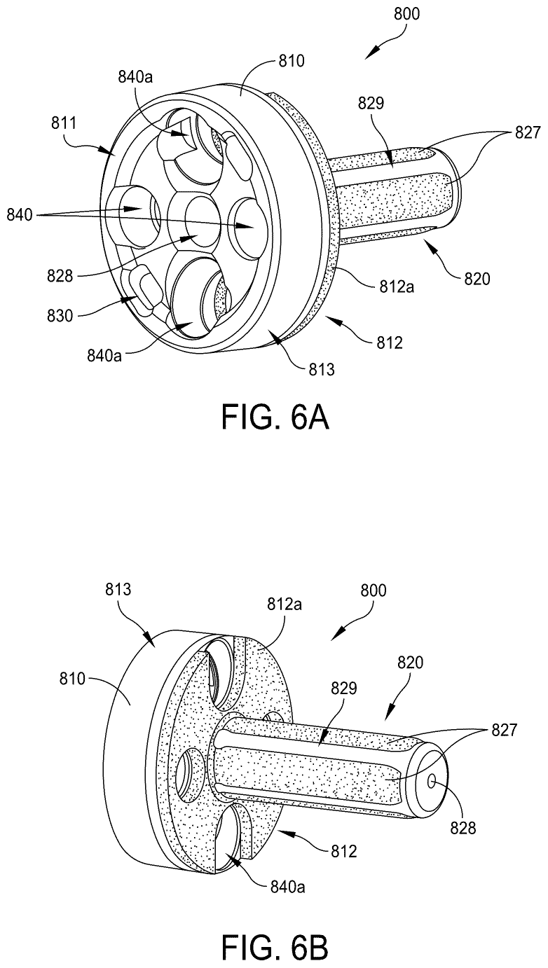

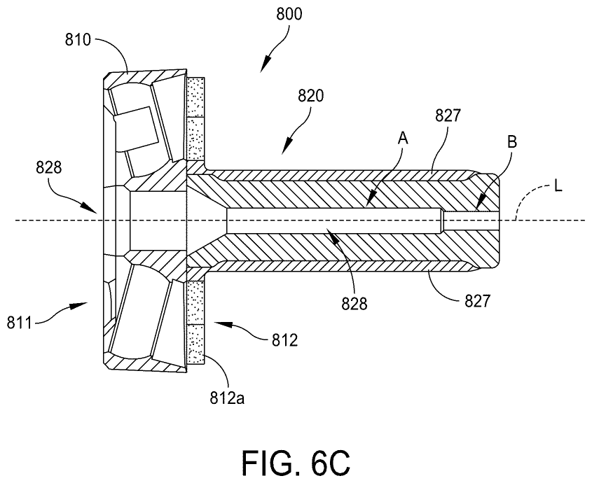

Referring to FIGS. 6A-6C, a prosthesis 800 for attachment to a glenoid according to another embodiment is provided. The prosthesis 800 comprises a baseplate body 810 that is a monolithic metallic structure. The baseplate body 810 comprises a distal surface 811, a proximal surface 812, and a post 820 extending from the proximal surface 812.

In this embodiment, the proximal surface 812 and the post 820 includes porous surface portions that act as scaffolds that facilitate bone ingrowth after the prosthesis 800 is implanted into a glenoid. As discussed below, the porous surface portions can also interact with modular bone augments that are made from bone grafts to facilitate bone ingrowth. For example, as shown in FIG. 6B, the proximal surface 812 can be provided with a porous surface portion 812a that is a surface layer. As shown in the illustrated example, the porous surface portion 812a can cover a substantial portion, if not all, of the proximal surface 812. The post 820 has an outer surface and comprises a plurality of porous surface portions 827 that are raised from the post's outer surface. Each of the raised porous surface portions 827 can extend along a substantial portion of the length of the post 820 separated by some space 829 as shown but in other embodiments, the specific pattern and shape of the plurality of porous surface portions 827 can be varied. The raised porous surface portions 827 cooperates with a modular bone augment that is slipped over the post 820 to provide orientational stability for the modular augment. In other words, the raised porous surface portions 827 cooperates with the mating surface of the modular bone augment to prevent the bone augment from rotating about the post 820. As will be discussed further below, the modular bone augment is configured with a mating surface that cooperates with the raised porous surface portions 827.

The porous surface portions 812a and 827 can be formed from the same metallic material and can be formed concurrently through additive manufacturing process. The post 820 is configured to be disposed in a complementary hole or a recess prepared in the glenoid to participate in affixing the prosthesis 800 to the glenoid.

In some embodiments, the prosthesis 800 can further comprise a plurality of fins (similar to the fins 130 illustrated with the prosthesis embodiment 100) that are radially extending from the post 820 at or near where the post meets the proximal surface 812. In some embodiments, the plurality of fins can be located where the post meets the proximal surface 812 and extend radially along the proximal surface. In some embodiments, the plurality of fins are located near where the post meets the proximal surface but do not touch the proximal surface 812.

In some embodiments, the prosthesis 800 can further comprise a plurality of openings 840, 840a provided between the post 820 and the baseplate body's periphery for receiving one or more bone screw to secure the prostheses 800 to the glenoid. Each of the plurality of openings 840, 840a extend through the baseplate body 810 from the distal surface 811 to the proximal surface 812. In some embodiments, some or each of the plurality of openings can be configured differently to accommodate different types of screws. For example, in the illustrated example, the two holes 840 are configured for receiving multi-directional screws without olives and the two holes 840a are configured for receiving multi-directional screws with olives.

In some embodiments, the prosthesis 800 can comprise one or more additional openings or recess 830 that are configured for receiving an instrument that is used to hold the prosthesis 800. In the illustrated example shown in FIG. 6A, the baseplate body 810 is provided with two oval shaped holes 830 for receiving ends of a clamp for example that can be used by a surgeon to hold the prosthesis during surgery.

Referring to FIG. 6C, in some embodiments, the post 820 can be configured with a hole or a channel 828 that assists in aligning an articular component, such as a glenosphere component 700 (see FIG. 5D) with the prosthesis 800 when the articular component is being attached to the baseplate body 810. The structure and function of the channel 828 is the same as the channel 128 provided in the post 120 of the prosthesis embodiment 100 that are described above.

Similarly, the channel 828 can be a blind hole that extends a predetermined depth into the post 820 so that the guide pin inserted into the portion A of the channel 828 extends out of the channel 828 at the distal surface 811 an appropriate amount and provide the guiding function for the articular component 700.

In some embodiments, the channel 828 can extend through the whole length of the post 820 so that the channel 828 is open at the distal surface 811 and at the proximal end of the post 820. In such embodiment, the channel 828 can be configured with two portions A and B where the portion A is the portion that opens at the distal surface 811 and receives the guide pin. The portion B can have a smaller diameter than the portion A so that the guide pin is too large to fit into the smaller diameter portion B. This configuration prevents the guide pin from exiting at the proximal end of the post 820 and into the patient's bone. Additionally, having the channel 828 open at both ends allow debris and fluids generated during the manufacturing process of drilling the channel 828 to be evacuated.

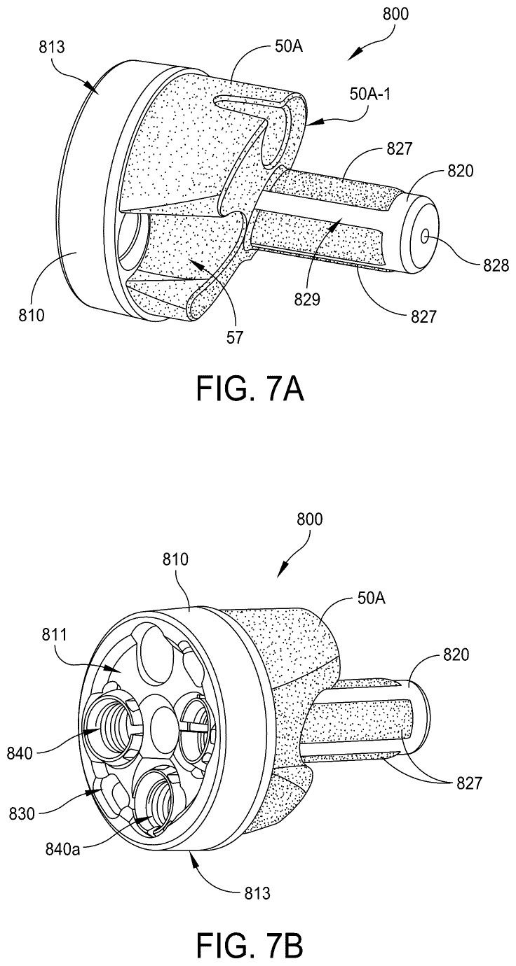

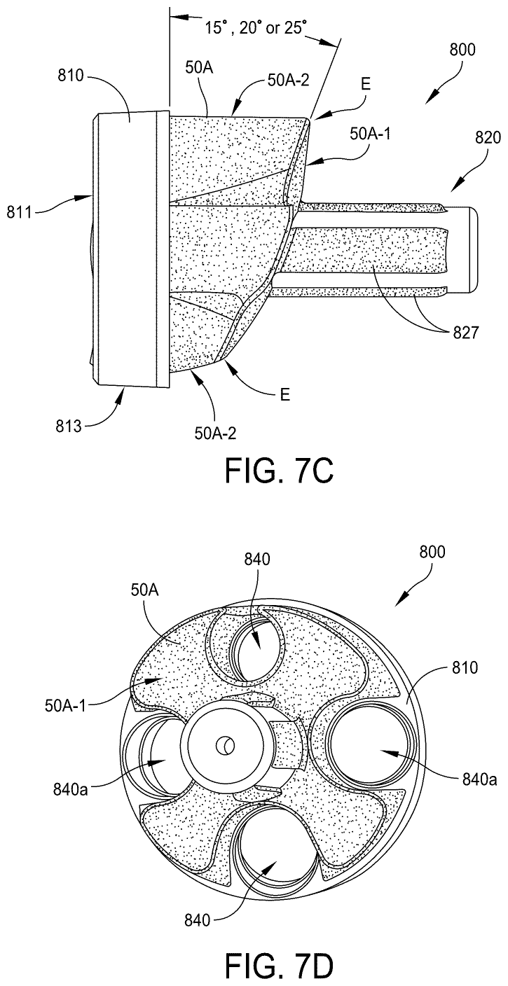

Referring to FIGS. 7A-7D, in some embodiments, the prosthesis 800 can further comprise a modular bone augment configured to be slipped over the post 820 and abut the porous surface portion 812a of the baseplate body 810. As discussed above, the modular bone augments can be formed of a bone graft material or a porous metallic material. In the example illustrated by FIGS. 7A-7D, the bone augment 50A is a porous metallic augment. The bone augment 50A is configured with a hole for receiving the post 820 so that the bone augment 50A can be slipped over the post 820 and contact the porous surface 812a of the baseplate body 810. The interior surface of the hole in the bone augment 50A for receiving the post 820 is configured to be the mating surface that cooperates with the raised porous surface portions 827 on the post 820 and provide orientational stability for the bone augment 50A by preventing or hindering the bone augment 50A from rotating about the post 820. For example, the interior surface of the hole in the bone augment 50A for receiving the post 820 can be configured to have a surface contour that is a negative (i.e. mirror image) of the raised porous surface portions 827 so that the bone augment 50A can be slipped over the post 820 by sliding in longitudinal direction parallel to the longitudinal axis L of the prosthesis 800 (see FIG. 6C). Once the bone augment 50A is slipped over the post 820, however, the raised porous surface portions 827 and the complementary mating surface of the bone augment 50A fit like gear teeth and the engagement between the raised porous surface portions 827 and the mating surface of the bone augment 50A prevents the bone augment 50A from rotating about the post 820.

The bone augment 50A can be configured with one or more cutouts 57 for accommodating the screws that would be inserted through the holes 840, 840a. The bone augment 50A has a bone-engaging surface 50A-1 whose surface contour is configured to have a shape that will appropriately fill a defective void in the glenoid. As mentioned previously, in some embodiments, the bone-engaging surface 50A-1 of the bone augment 50A can be configured with a patient-matched surface contour for the condition of a particular patient's glenoid.

In some embodiments, the post 820 can be configured with a hole or a channel 828 that can assist in aligning an articular component with the prostheses 800 when the articular component is being attached to the baseplate body 810. The channel 828 extends from the distal surface 811 into the post 820 along the longitudinal axis L of the post 820. The structure of the channel 828 can be the same as the channel 128 in the post 120 of the prosthesis embodiment 100 and the channel 828 of the prosthesis 800 receives a guide pin in the same manner as described above for the channel 128 in the post 120.

According to another aspect of the present disclosure, FIGS. 7A-7D also illustrate an example of another embodiment of a prosthesis for attachment to a glenoid that incorporates an integrally formed metallic bone augment. According to such embodiment, the prosthesis 800 comprises a baseplate body 810 that is a monolithic metallic structure and comprises: a distal surface 811, and a proximal surface 812 opposite from the distal surface. In this embodiment, the proximal surface 812 comprises a bone augment portion 50A and a post 820 extending from the bone augment portion 50A. In this embodiment, the bone augment portion 50A and the post 820 are integrally formed as part of the monolithic metallic structure of the baseplate body 810.

The metallic bone augment portion 50A of the proximal surface 812 extends away from the distal surface 811 and defines a bone-engaging surface 50A-1 that is spaced apart from the distal surface 811. The post 820 comprises an outer surface and a plurality of porous surface portions 827 that can be raised from the outer surface of the post. In some embodiments, the porous surface portions 827 can be flush with the outer surface of the post 820.

In some embodiments of the prosthesis 800 having the integrally incorporated metallic bone augment portion 50A, the bone augment portion 50A is a porous structure. In some embodiments, the plurality of porous surface portions 827 can be formed as structures that are raised from the surface of the post 820 and extend longitudinally along a substantial portion of the length of the post 820. Each of the raised porous surface portions 827 can extend along a substantial portion of the length of the post 820 separated by some space 829 as shown but in other embodiments, the specific pattern and shape of the plurality of porous surface portions 827 can be varied.

Referring to FIG. 7C, the bone-engaging surface 50A-1 is oriented at an oblique angle with respect to the distal surface 811 of the baseplate body 810. In some embodiments, the oblique angle can be between 10 and 30 degrees. In some embodiments, the oblique angle can be between 15 and 25 degrees.

In some embodiments, the bone-engaging surface 50A-1 is a non-flat surface. In some embodiments, the bone-engaging surface 50A-1 is a spherical surface. In some embodiments, the spherical surface has a radius of curvature that is within a range of 30 mm to 50 mm. In some embodiments where the bone-engaging surface 50A-1 is a spherical surface, the spherical surface 50A-1 is oriented at an oblique angle between 10 and 30 degrees with respect to the distal surface 811. In some embodiments the oblique angle can be between 15 and 25 degrees.

Referring to FIG. 7C, for the bone-engaging surface 50A-1 that has a spherical curvature, the surface's obliquely angled orientation is defined as the angular orientation of the edge E with respect to the distal surface 811 when viewed from the side as shown in FIG. 7C. The edge E is defined as the edge where the bone-engaging surface 50A-1 intersects the side surface 50A-2 of the bone augment portion 50A.

In some embodiments of the prosthesis 800 having the integrally incorporated metallic bone augment portion 50A, a plurality of openings 840, 840a can be provided between the post 820 and the baseplate body's periphery, where each of the plurality of openings extend through the baseplate body 810 from the distal surface 811 to the proximal surface 812 for receiving one or more screw. In some embodiments, the prosthesis 800 can further comprise one or more additional openings or recess 830 that are configured for receiving an instrument that is used to hold the prosthesis 800. In the illustrated example shown in FIG. 7B, the baseplate body 810 is provided with two oval shaped holes 830 for receiving ends of a clamp for example that can be used by a surgeon to hold the prosthesis during surgery

In some embodiments, the post 820 has a longitudinal axis and can comprise a channel 828 that extends into the post from the distal surface 811 and extends along the longitudinal axis L of the post. The structure of the channel 828 can be the same as the channel 128 in the post 120 of the prosthesis embodiment 100 and the channel 828 of the prosthesis 800 receives a guide pin in the same manner as described above for the channel 128 in the post 120.

In some embodiments, the porous metal structures mentioned in the present disclosure are additively fabricated (e.g. 3D printed) metallic structures. An example of such porous metallic material is ADAPTIS™ material by Wright Medical Group N.V.

Referring to FIG. 5D, each of the prosthesis embodiments 100, 100A, 200, 300, 400, 500, and 800 can further comprise an articular component 700 configured to removably couple to the baseplate body 110, 210, 310, 410, 510, 810. The articular component 700 can be an anatomic articular component replacing the anatomic glenoid bearing surface or a reverse articular component. In FIG. 5D, an example of the articular component 700 shown is a reverse articular component comprising a convex articular surface 710. The articular component can be configured with a recess 720 that is configured to fit onto a baseplate body 110, 210, 310, 410, 510, or 810 and form a friction lock engagement with the respective peripheral surface 113, 213, 313, 413, 513, and 813 of the baseplate body 110, 210, 310, 410, 510, and 810. In some embodiments, the recess 720 can have an interior surface and the interior surface and the corresponding peripheral surface 113, 213, 313, 413, 513, and 813 on the respective baseplate body 110, 210, 310, 410, 510, 810 can be configured to form a machine taper system. In other words, each of the peripheral surfaces 113, 213, 313, 413, 513, 813 form the conical male member of the machine taper system and the interior surface of the recess 720 form the corresponding female socket member of the machine taper system. An example of such machine taper system can have Morse taper surfaces. The articular component can be formed of a metal, a polymer, or a combination of metal and polymer depending on the particular application.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

1.-69. (canceled)

70. A prosthesis for attachment to a glenoid comprising:
a baseplate body that is a monolithic metallic structure and comprises:
a distal surface and a proximal surface opposite from the distal surface, wherein the proximal surface comprises a bone augment portion that extends away from the distal surface and defines a bone-engaging surface that is spaced apart from the distal surface;
a post extending from the bone augment, wherein the post comprises an outer surface and a plurality of porous surface portions.

71. The prosthesis of claim 70, wherein the plurality of porous surface portions are raised from the outer surface of the post.

72. The prosthesis of claim 70, wherein the bone augment portion is a porous structure.

73. The prosthesis of claim 70, wherein the post has a length and the raised plurality of porous surface portions extend longitudinally along a substantial portion of the length of the post.

74. The prosthesis of claim 70, wherein the bone-engaging surface is oriented at an oblique angle with respect to the distal surface of the baseplate body.

75. The prosthesis of claim 74, wherein the oblique angle is between 10 and 30 degrees.

76. The prosthesis of claim 74, wherein the oblique angle is between 15 and 25 degrees.

77. The prosthesis of claim 74, wherein the bone-engaging surface is a non-flat surface.

78. The prosthesis of claim 77, wherein the bone-engaging surface is a spherical surface. Preliminary Amendment

79. The prosthesis of claim 78, wherein the spherical surface has a radius of curvature that is within a range of 30 mm to 50 mm.

80. The prosthesis of claim 78, wherein the oblique angle is between 10 and 30 degrees.

81.-87. (canceled)
